(19) United States
(12) Patent Application Publication
(10) Pub. No.: US 2011/0124867 A1
(43) Pub. Date: May 26, 2011

(54) Process and intermediates for the Synthesis of heterocyclic Substituted Piperazines with CXCR3 Antagonist Activity

(75) Inventors: Frank Xing Chen, Plainsboro, NJ (US); Timothy D. Cutarelli, Clinton, NJ (US); Xiayong Fu, Edison, NJ (US); Xian Liang, Monmouth Junction, NJ (US); Timothy L. McAllister, (US); Robert K. Orr, Cranford, NJ (US); Jianguo Yin, Plainsboro, NJ (US); Kelvin H. Yong, Lyndhurst, NJ (US); Man Zhu, Clark, NJ (US)

(73) Assignee: Schering Corporation, Kenilworth, NJ (US)

(21) Appl. No.: 12/809,101

(22) PCT Filed: Dec. 16, 2008
(86) PCT No.: PCT/US08/86949

§ 371 Date: Jun. 18, 2010

Related U.S. Application Data
(60) Provisional application No. 61/008,233, filed on Dec. 18, 2007.

(51) Int. Cl. C07D 413/14; C07D 401/14; C07D 403/04; C07D 241/04; C07D 211/44

(52) U.S. Cl. 544/357; 544/389; 546/220

(57) ABSTRACT

The present invention relates to novel processes for the preparation of the compound of the Formula A45:

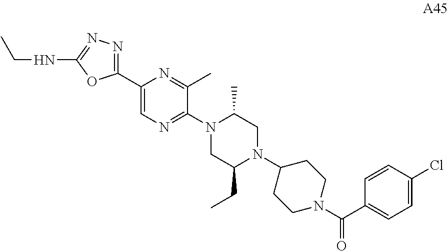

or a physiologically acceptable salt, solvate or prodrug thereof, which has utility, for example, as a pharmaceutically active compound with CXCR3 antagonist activity, and to novel intermediates useful in the synthesis thereof.

### CROSS REFERENCE TO RELATED APPLICATIONS

The present application is based on and claims the priority of U.S. Provisional Application 61/008,233 filed Dec. 18, 2007, which application is incorporated by reference in its entirety as if fully set forth herein.

### FIELD OF THE INVENTION

This application relates to novel processes for the preparation of heterocyclic substituted piperazines, which have utility, for example, as pharmaceutically active compounds with CXCR3 antagonist activity, and to novel intermediates useful in the synthesis thereof.

### BACKGROUND OF THE INVENTION

Identification of any publication in this section or any section of this application is not an admission that such publication is prior art to the present invention.

CXCR3 antagonists have been identified as being useful in the provision of palliative, curative and prophylactic therapies for a number of diseases and medical conditions, non-limiting examples of which include inflammatory conditions, for example, psoriasis and inflammatory bowel disease, autoimmune disease, for example, multiple sclerosis, rheumatoid arthritis, fixed drug eruptions, cutaneous delayed-type hypersensitivity responses, type I diabetes, viral meningitis and tuberculoid leprosy. CXCR3 antagonist activity has also been indicated as a therapy for tumor growth suppression as well as graft rejection, for example, allograft and zenograft rejection.

The compound of Formula A45 has been found to have useful CXCR3 inhibitor activity. The synthesis of the compound of Formula A45 has been described in published U.S. patent application Ser. No. 11/353,641 (hereinafter “the '641 application,”) filed Feb. 14, 2006 and published under Publication No. 2006/0276479 on Dec. 7, 2006, which is incorporated herein in its entirety by reference.

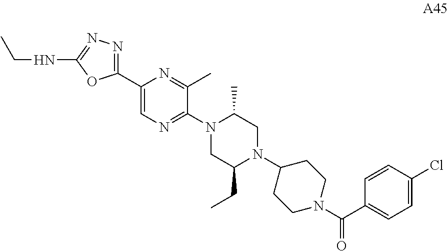

As described in the '641 application in preparative Examples 21-28 (see the '641 application in numbered paragraphs [0428] to [0448], inclusive), the compound of Formula A45 can be prepared in accordance with the process described in Scheme I.

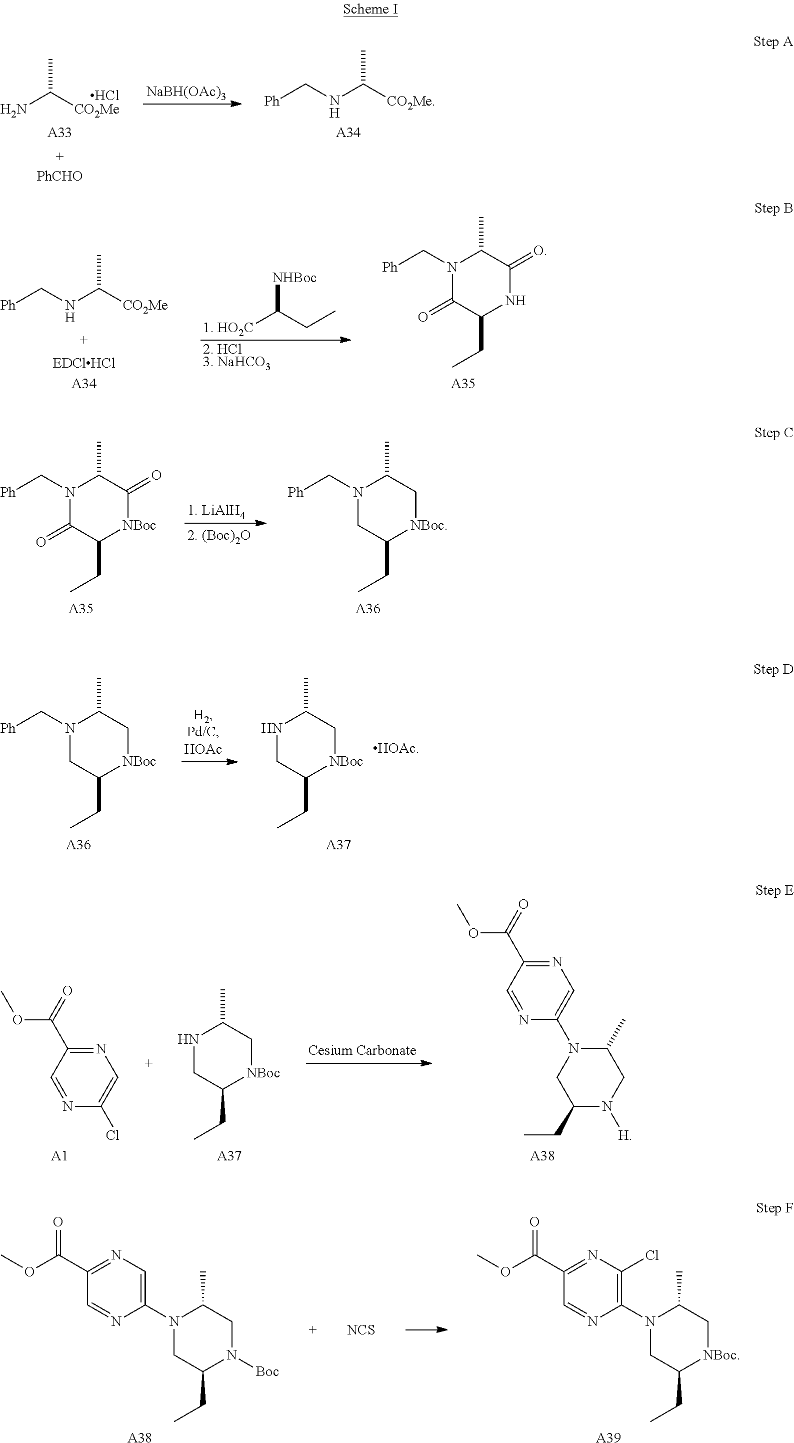

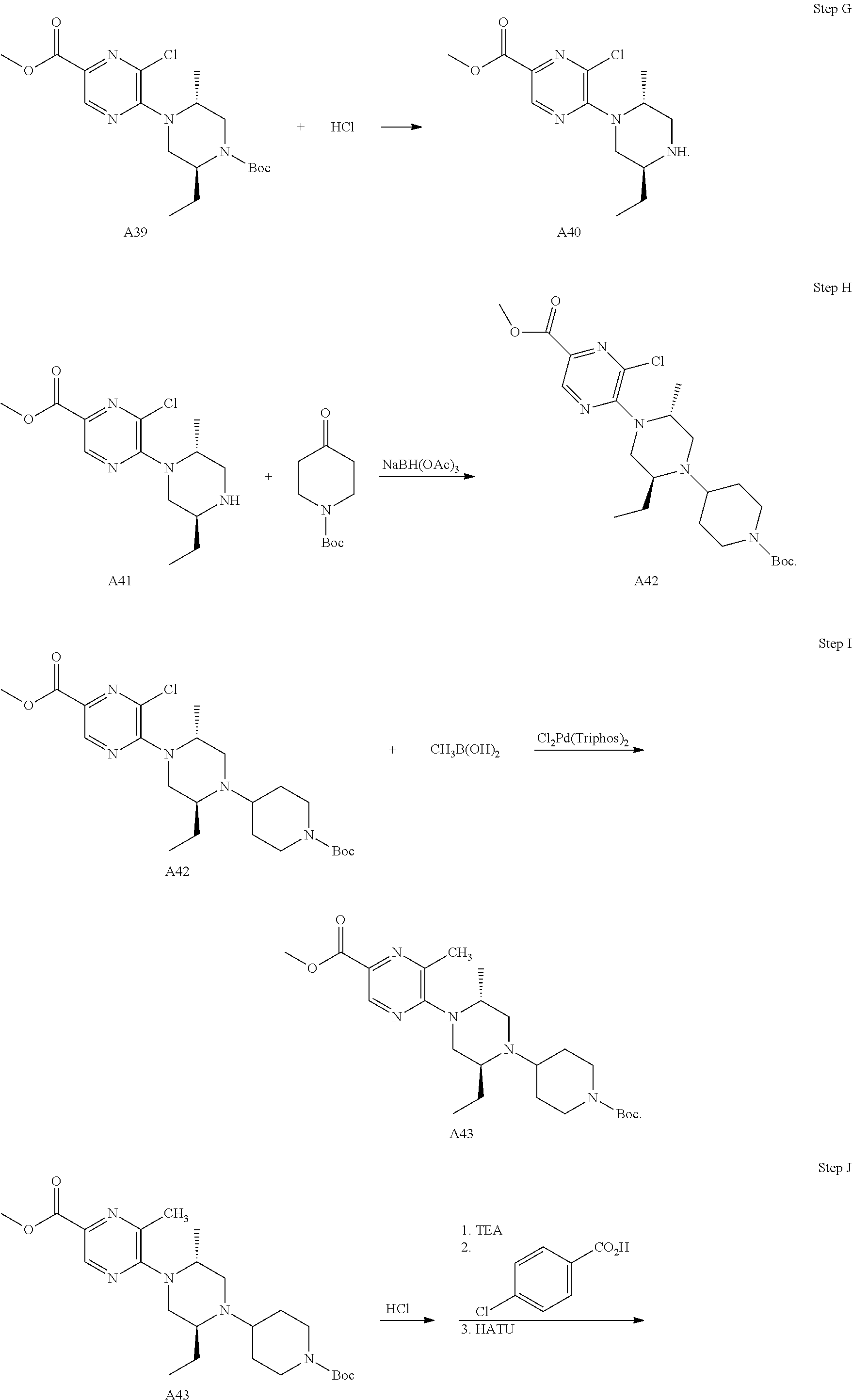

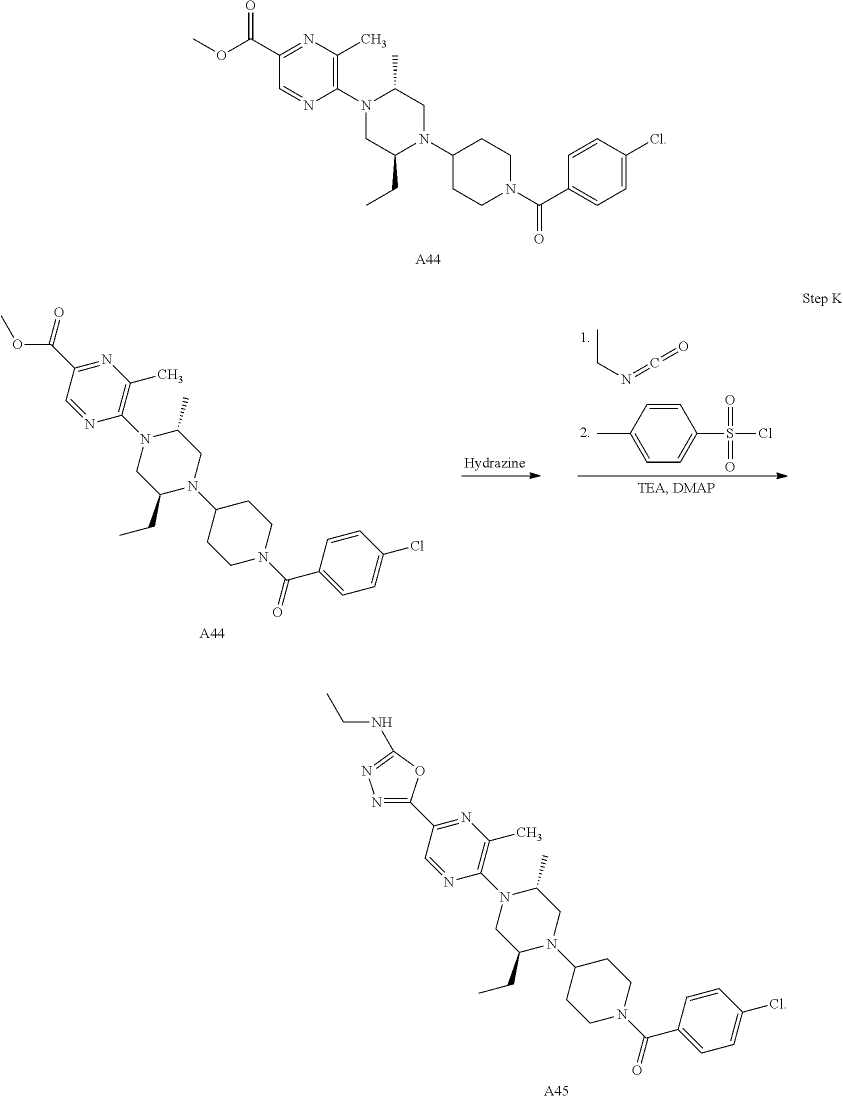

Scheme I involves 11 individual reaction sequences, almost half of which have multiple steps in the sequence and provides the compound A45 in an overall yield of 8% based upon the amount of the compound of Formula A33 utilized in the reaction. Moreover, the synthetic scheme described in the '641 application requires numerous intermediates be purified using chromatographic separation and purification techniques, which are impractical for preparing commercial-scale quantities of the compound.

### OBJECTIVES AND SUMMARY

In view of the foregoing, what is needed is a synthetic scheme useful for preparing CXCR3 inhibitor compounds of Formula A45, and their salts, which utilizes safer materials and provides a reaction scheme affording practical scale up to a batch size suitable for commercial scale preparation. These and other objectives and/or advantages are provided by the present invention.

One aspect of the present invention is a novel process in accordance with Scheme II for making the compound of Formula A45.

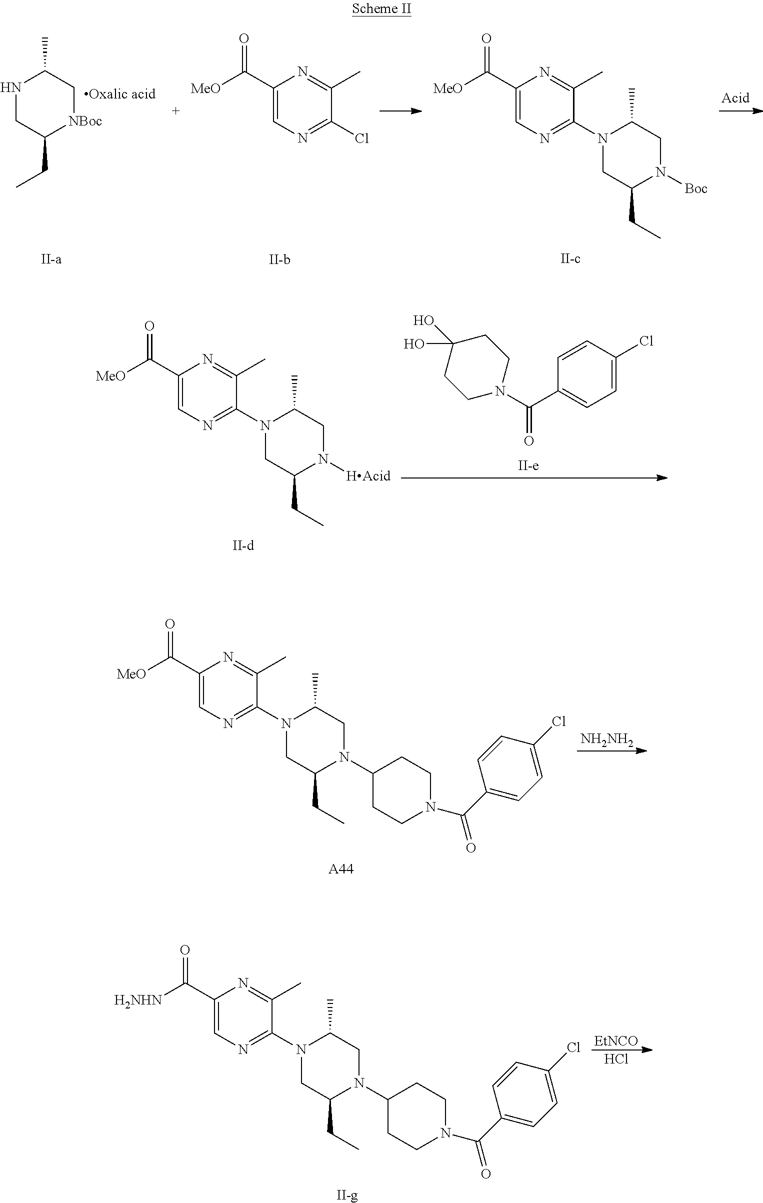

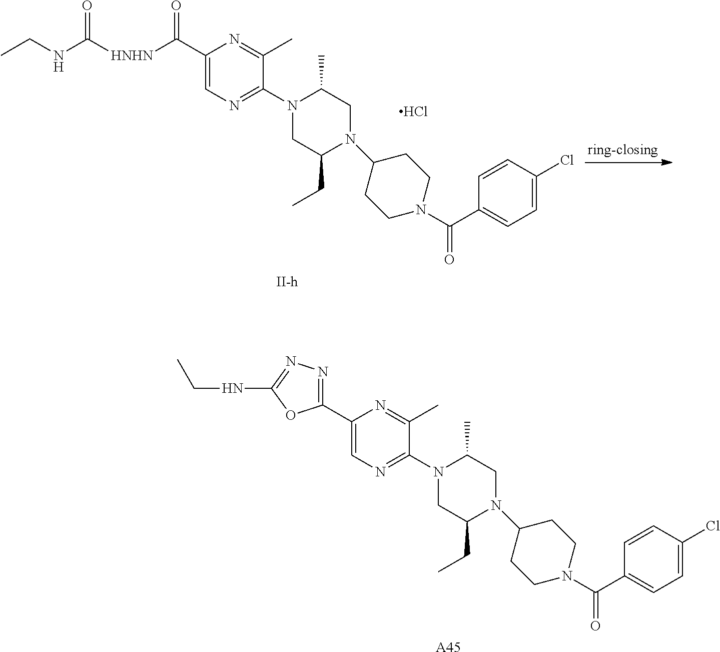

the process comprising:
(i) providing the oxalate salt compound of Formula II-a;
(ii) coupling the free-base form of the oxalate salt obtained in Step “i” with the compound of Formula II-b to form the compound of Formula II-c;
(iii) deprotecting the protected nitrogen of the compound of Formula II-c by treatment with acid, thereby forming the salt compound of Formula II-d;
(iv) coupling the II-d salt compound prepared in step “iii” with the compound of Formula II-e in the presence of sodium triacetoxyborohydride to form the compound of Formula A44;
(v) reacting the compound of Formula A44 from Step “iv” with hydrazine, to form the compound of Formula IIg, and optionally isolating the compound of Formula IIg;
(vi) reacting the compound of Formula IIg sequentially with Ethylisocyanate (EtNCO), then HCl to precipitate the compound of formula of Formula II-h; and
(vii) cyclizing the hydrazide group of the compound of Formula II-h to form the compound of A45.

In some embodiments, it is preferred to carry out the provision of the oxalate salt compound of Formula II-a in accordance with the process depicted in Scheme III.

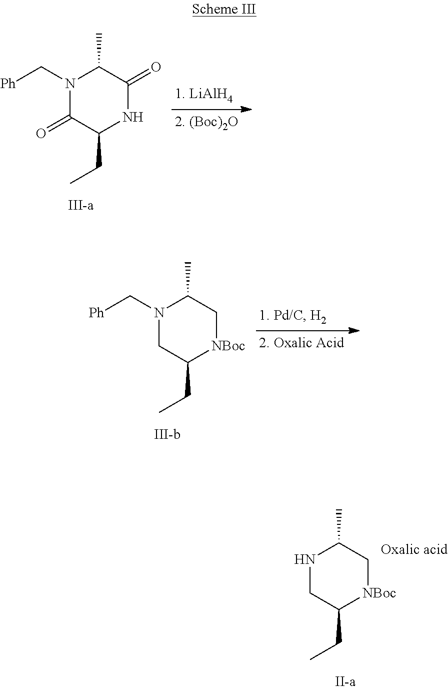

this process comprising:
(i) providing the compound of the Formula III-a;
(ii) reacting the compound of the Formula III-a with lithium aluminum hydride and then di-tert-butyl dicarbonate to form the compound of the Formula III-b; and
(iii) hydrogenating the compound of the Formula III-b and treating the resulting product with oxalic acid to yield the oxalate salt compound of the Formula II-a.

In some embodiments, the compound of the Formula III-a is preferably provided in accordance with the process depicted in Scheme IV:

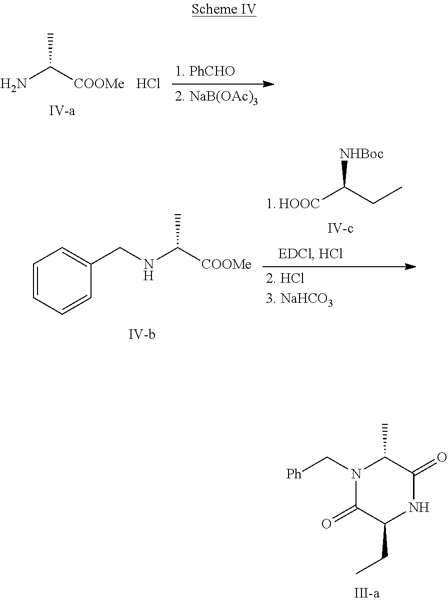

this process comprising:
(i) providing the compound of the Formula IV-a;
(ii) coupling the compound of the Formula IV-a or a salt thereof with benzaldehyde (PhCHO) to form the compound of the Formula IV-b;
(iii) reacting the compound of the Formula IV-b with a compound of the Formula IV-c; and
(iv) deprotecting then cyclizing the deprotected product to form the compound of the Formula III-a.

In some embodiments it is preferred to obtain the compound of Formula III-a by distilling off the organic solvent containing the product and recrystallizing the compound of Formula III-a contained in the residue from a 60:40 mixture of tertiarybutyl methyl enther:heptane to obtain the deprotected compound of Formula IIIa.

In some embodiments, the compound of the Formula II-e is prepared in accordance with the process depicted in Scheme V:

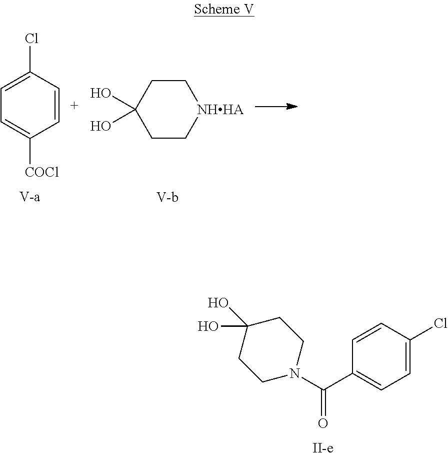

this process comprising reacting the compound of the Formula V-a with a compound of the Formula V-b. In some embodiments it is preferred to isolate the compound of Formula II-e as a crystalline solid directly from the reaction mixture by neutralizing the reaction mixture at a temperature sufficiently below ambient to precipitate the product, thereby providing the compound as a hydrate in higher purity and greater yield than is reported in literature preparation of similar compounds, for example, as reported in Bioorganic & Medicinal Chemistry Letters 13(14) p. 2303, (2003).

Further aspects of the present invention include novel intermediates useful in the foregoing inventive processes, these intermediates being the compounds of the Formulae II-c II-g and II-h.

The present invention also includes the novel physiologically tolerated salt compound of the Formula II-1.

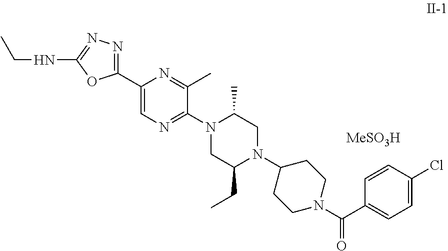

### DETAILED DESCRIPTION OF THE INVENTION

As mentioned above, and described in the '641 application, the compound of Formula A45 and physiologically tolerated salts, solvates and prodrugs thereof have CXCR3 inhibiting properties, for example, the mesylate salt. The present application provides a commercial-scale synthesis of these compounds.

Compared with the preparation process for the Compound A45 described in the '641 application, the inventive process affords a number of improvements from the standpoints of utilization of substrates, utilization of intermediates, convergence and handling. The overall result is a more efficient and streamlined process that can be scaled-up to provide a commercial-scale synthesis of Compound A45 and physiologically tolerated salts, solvates and prodrugs thereof.

In the inventive process, use is advantageously made of the oxalate salt compound of the Formula II-a. In the process described in the '641 application, the Compound A36 is reacted with glacial acetic acid to yield an acetic acid salt Compound A37 after workup as a viscous oil. In contrast, the Compound II-a is obtained according to an embodiment of the inventive process as a crystalline solid, which greatly improves handling and further processing. Moreover, in the process of the present invention, the compound of Formula III-a, from which the compound of Formula IIa is prepared, surprisingly, isolation of the compound of Formula III-a from the reaction mixture by evaporating the reaction solvent, then crystallizing the compound form a 60:40 (vol) mixture of tertiarybutyl methyl ether (MTBE):heptane provides the compound in a highly purified form which eliminates the chromatography steps needed in previous reported reaction to provide suitably pure amounts of the compound of Formula II-a. This has the benefit of providing the compound of Formula 0 kb in a suitably pure for use in the preparation of the compound of Formula II-a, and has the added benefit of increasing the yield of the compound of Formula II-a from the compound of Formula III-a.

In the inventive process, use is also advantageously made of the methylated piperazine of the Formula II-b. In the process described in the '641 application, the Compound A37 is reacted with the Compound A1, which is not methylated on the piperazine. This necessitates two additional process steps, which are described in Preparative Example 23 and Preparative Example 26 in the '641 application, in order to introduce the methyl group to the piperazine moiety. In the first process step, a chlorine atom is introduced to the piperazine moiety by reacting the Compound A38 with N-chlorosuccinimide. In the second process step, this chlorine atom, which is still present in Compound A42, is converted to the required methyl group by reacting the Compound A42 with methylboronic acid. In contrast, by making use of the methylated piperazine of the Formula II-b, which is a known and readily available chemical, the inventive process completely avoids these unnecessary steps, resulting in a simpler, more streamlined process characterized by better utilization of substrate.

According to the present invention, the free base of the Compound II-a and the Compound II-b are reacted to form the Compound II-c. Surprisingly, the inventors have found that the use of a crystalline oxalic acid salt form of 2-ethyl-5-methyl piperazine (the compound of Formula II-a) provides an intermediate which is easier to handle than the acetic acid salt intermediate described in the '641 application, simplifying scale up of the process to a size suitable for use in preparing commercial quantities of the intermediate.

In the inventive process, the compound of the Formula II-c is deprotected by treating with an acid to form a salt form of the compound of the Formula II-d. The acid can be any suitable acid, and is preferably selected from mineral acids such as hydrochloric, sulfuric, nitric and phosphoric acids, organic acids such as sulfonic acids, e.g. benzenesulfonic (besylic), p-toluenesulfonic (PTSA, tosylic), methanesulfonic (MSA, mesylic) and trifluoromethanesulfonic (triflic) acids; and carboxylic acids e.g., formic, acetic, proprionic, benzoic, citric, tartaric, maleic, fumaric, succinic and malic acids, just to name a few. In a preferred embodiment, the acid is hydrochloric acid. In an especially preferred embodiment, the deprotection with hydrochloric acid is carried out by the addition of 2-isopropanol hydrochloride solution.

When the compound of the Formula II-c is deprotected with hydrochloric acid, the resulting hydrochloric acid addition salt has the Formula II-d-1:

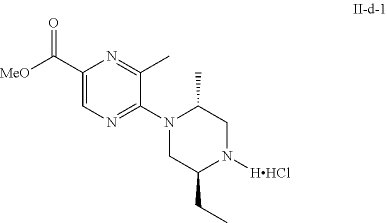

The reaction of the Compound II-d-1 with the Compound II-e to form the Compound A44 is preferably carried out in the presence of NaBH(OAc)₃.

The use of the Compound II-e represents a significant improvement over the process described in the '641 application, wherein the use of N-Boc-4-piperidone in a step-wise build up of the molecular core structure necessitated two additional steps, a deprotection step and coupling with 4-chlorobenzoic acid. In a preferred embodiment, the Compound II-e is advantageously prepared according to Scheme V above, and especially as a crystalline monohydrate. The pure monohydrate precipitates directly from the reaction mixture in very high yield, thus significantly simplifying the entire process.

The Compound A44 is then reacted with hydrazine, preferably aqueous hydrazine in an alcohol, especially methanol, to form the Compound II-g.

In some embodiments of the inventive process, the conversion of the Compound II-d-1 to the Compound II-g is accomplished by a process which is simplified when compared to that described in the above-mentioned '641 application. In the process described in the '641 application, the Compound A41 is converted to the Compound A44 in a stepwise procedure which includes multiple isolations of solid intermediate products which are operationally difficult to perform in a large scale process, often utilizing chromatography to accomplish the isolation at the end of each step. In contrast, the present inventive process affords the advantage of converting the Compound II-d-1 to the Compound II-h without the need to isolate intermediate products, thereby, again, making the overall process more efficient and streamlined.

Alternatively, in some embodiments of the inventive process, the intermediate Compound II-g may be isolated and characterized, if desired.

In a preferred embodiment, the Compound II-g, whether isolated or not, is converted to the Compound II-h by reacting with Ethylisocyanate (EtNCO), followed by reaction with hydrochloric acid, which causes the Compound II-h to precipitate out of the reacting mixture. Optionally, other mineral acids can be employed to precipitate the Compound of Formula II-h to precipitate out, for example other mineral acids, for example, phosphoric acid. In some embodiments it is preferred to prepare the compound of II-h as the phosphate salt to insure high purity of the intermediate and provide a precipitate which has acceptable filtration and handling properties

The hydrazide functionality of the Compound II-h is then cyclized to form the Compound A45. In some embodiments it is preferred to carry out the cyclization reaction in the presence of a catalytic amount of dimethylaminopyridine (DMAP), particularly in the presence of potassium carbonate and 4-chlorobenzenesulfonylchloride, or, alternatively, in the presence of triethylamine and diethylphosphoric chloride. In some embodiments the cyclization reaction is carried out in a solvent selected from tetrahydrofuran (THF) and mixtures of THF and acetonitrile, preferably, the cyclization reaction is carried out in tetrahydrofuran as solvent. In some embodiments it is preferred to convert the compound of Formula II-h into a free-base form, for example, by treating it with potassium carbonate, prior to cyclizing it to yield the compound of Formula A45. Without wanting to be bound by theory, it is believed that such a modification results in a reaction utilizing a higher percentage of the intermediate compound of Formula II-h and which runs to completion in less time.

Once the compound of the Formula A45 is obtained, it can be converted, if desired, to a physiologically tolerated salt, solvate or prodrug thereof. In the context of the present invention, “physiologically tolerated salts” are all those physiologically tolerated salts contemplated in the description of the '641 application. Especially, the present invention extends to the preparation of physiologically tolerated acid addition salts of the compound of Formula A45. Acids that are generally considered suitable for the formation of physiologically tolerated salts from basic pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Intl. Union of Pure and Applied Chemistry, pp. 330-331. These disclosures are incorporated herein by reference thereto. Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, alkylsulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like. In an especially preferred embodiment, the acid addition salt is a methylsulfonate having the Formula II-1. Surprisingly, the methylsulfonate salt uniquely provides a stable crystalline solid which has higher aqueous solubility and thereby improved biological activity when compared to other salts.

Likewise, “physiologically tolerated solvates” are all those physiologically tolerated solvates contemplated in the description of the '641 application. The term “solvate,” as used therein and incorporated herein by reference to the '641 application, means a physical association of a compound of the invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O. In general, the solvated forms are equivalent to the unsolvated forms and are intended to be encompassed within the scope of this invention.

“Physiologically tolerated prodrugs” are all those physiologically tolerated prodrugs contemplated in the description of the '641 application. The term “prodrug,” as employed therein and incorporated herein by reference to the '641 application, denotes a compound that is a drug precursor which, upon administration to a subject, undergoes chemical conversion by metabolic or chemical processes to yield a compound of Formula I or a salt and/or solvate thereof. A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

Exemplary embodiments of the present invention will now be described in greater detail with reference to the following non-limiting examples.

### EXAMPLES

The following solvents and reagents may be referred to by their abbreviations in parenthesis:

sodium bistrimethylsilylamide: NaHMDS
triethyl amine: TEA
trifluoro acetic acid: TFA
tertiary-butoxycarbonyl: t-BOC
tetrahydrofuran: THF
lithium bis(trimethylsilyl)amide: LiHMDS
mole: mol.

The compounds of the Formula A45 and the Formula II-1 are prepared in accordance with Scheme VI.

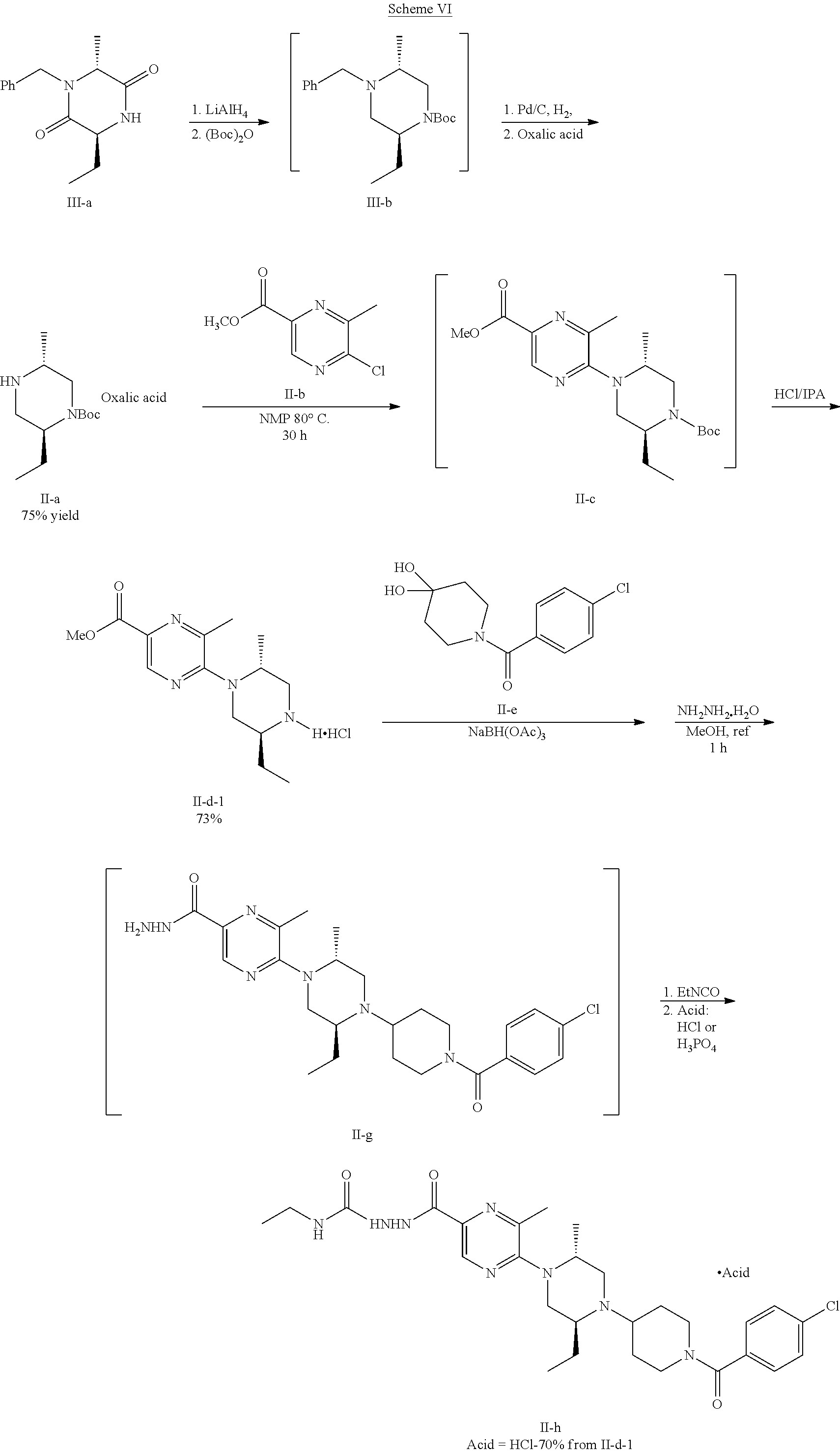

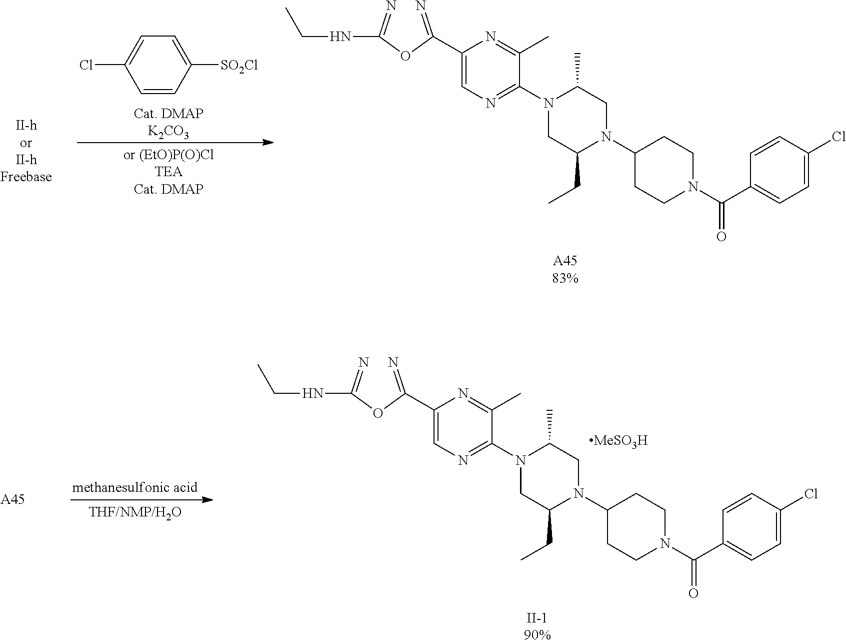

### Preparation of Compound II-a

To a solution of Compound III-a (50 g) in 250 ml of THF was added a solution of lithium aluminum hydride in THF (2.4M) (170 ml) slowly at a temperature below 25° C. The reaction mixture was then heated to 65° C. for 2 hours. The mixture was cooled to 10° C. and water (25 ml) was added very slowly at a temperature below 25° C. over one hour. The resulting mixture was agitated for 1 hour at 25° C. The reaction mixture was then quenched by slowly adding sodium hydroxide solution (25%, 50 ml) over one hour. After the mixture was agitated for one additional hour, di-tert-butyl dicarbonate, 70% in THF (68 ml) was added over 10 minutes at a temperature below 30° C. The resulting mixture was stirred one hour and ammonium hydroxide, ˜28% (5 ml) was added over 30 minutes. The mixture was then filtered and the cake was washed with THF (3×200 ml). The filtrate and washes were combined and concentrated to 150 ml under vacuum. After addition of isopropyl alcohol (250 ml), the mixture was concentrated again to 150 ml under vacuum. The resulting solution is transferred into a hydrogenator and acetic acid (10 ml), Palladium on carbon (5 g) were charged into the reactor. The mixture was agitated under hydrogenation (90 psi.) for about 10 hours or until the reaction was completed. The mixture was filtered and the filtrate was transferred to another flask, which contained oxalic acid (3.8 g). The mixture was concentrated to 200 ml and maintained at 40° C. TBME (200 ml) is charged into the mixture at 40° C. and slowly cooled to room temperature. After mixture was agitated for another hour, the solids were collected by filtration and the wet cake was washed with TBME. The wet cake was dried under vacuum at 50° C., to give 48.0 g of white solids (74.4%). ¹H NMR (400 MHz, in CD₃OD): 4.68 (m, 1H); 3.94 (d, 1H); 3.65 (m, 1H); 3.39 (d,d, 1H), 3.35 (d, 1H), 3.10 (d, 1H), 1.80 (m, 1H), 1.60 (m, 1H); 1.50 (s, 9H); 1.38 (d, 3H), 0.93 (t, 3H); ¹³C NMR (400 Mhe, CD₃OD): 166.9, 157.0, 82.5, 51.6, 49.3, 41.6, 41.27, 28.9, 23.6, 14.1, 11.0.

### Preparation of Compound II-d-1

Water (500 ml) and Compound II-a (85.0 g) were changed into a flask and the mixture was agitated for 10 to 20 minutes. Potassium Phosphate (127 g) and toluene (500 ml) were charged into the reaction. The batch was agitated until all solids were dissolved. The aqueous was split and the organic phase was concentrated to about 170 ml or less if possible under vacuum. 1-Methyl-2-pyrrolidinone (NMP) (204 ml) was charged to the batch, which was further concentrated to about 300 ml under vacuum. The batch was cooled to below 0° C. To the batch, Compound II-b (50 g) and N,N diisopropylethylamine (60 ml) were charged. The batch was heated to 90° C. for about 17 hours until the reaction was completed. The batch was concentrated under vacuum at 80° C. until no distillate came out. The batch was cooled to 20° C. Water (255 ml) and TBME (510 ml) were added into the batch. The mixture was agitated and phases were separated in a separation funnel. The organic layer was washed again with 250 ml of water. The combined aqueous layer was back extracted with 200 ml of TBME. The combined organic layer was washed with 0.5N HCl (100 ml). The batch was concentrated at vacuum until 255 ml. TBME (510 ml) was charged into the batch and concentrated to 250 ml. After cooling to 0-10° C., a solution of 2-isopropanol hydrochloride solution (5-6N, 170 ml) was added under 25° C. and the solution was agitated at 15-25° C. for 20 hours. TBME (255 ml) was charged into the batch and agitated at 25° C. for 2 hours. The batch was filtrated and washed with a mixture of TBME/2-Isopropanol. (200 ml, 2/1 v/v). The wet cake was dried at vacuum oven at 50° C. to give 65.0 g (77%) of the white solid. ¹H NMR (400 MHz in CD₃OD): 8.86 (S, 1H), 4.83 (S, 2H), 3.94 (m, 4H); 3.53 (m, 3H); 3.24 (t, 1H); 2.99 (dd, 1H); 2.66 (S, 3H), 1.82 (m, 2H); 1.11 (m, 6H). ¹³C NMR (400 Mhe, CD₃OD): δ 165.3; 160.4; 152.4;143.3; 139.2′ 58.3; 53.8; 53.8; 51.5; 49.9, 25.1; 21.4; 17.1, 10.6.

### Preparation of Compound II-h (Acid=HCl) From the Compound of II-d-1

Compound II-d-1 (89.3 g, 1.0 eq) and Na₃PO₄ (69.7 g) were added to the reaction vessel followed by water (446 ml) and MeOAc (446 ml). The mixture was heated to 35-45° C. until all solids were dissolved and clean phase split was observed. The aqueous phase was split and the organic was washed with 20% NaCl solution (446 ml). The mixture was concentrated under vacuum at 40° C. to a volume of about 250 ml and dissolved with 450 ml of THF and again concentrated under vacuum at 40° C. to a volume of about 250 ml. The mixture was then dissolved with 900 ml of THF and transferred to another reactor containing Compound II-e and agitated until dissolved. The mixture was concentrated under vacuum at 40° C. to a volume of about 250 ml. The remaining solution was dissolved with 600 ml of THF and again concentrated under vacuum at 40° C. to a volume of about 250 ml. The mixture was dissolved with 600 ml of THF. The mixture was transferred to another reactor containing NaBH(OAc)₃ (96.5 g) and NaSO₄ (202 g). The mixture was agitated at 20-30° C. for about 18 h. When the reaction was complete, water (900 ml) was added as quench and the batch was heated to 35-45° C. to dissolve all solids. The aqueous phase was separated in a separation funnel. The aqueous phase was back-extracted with EtOAc (268 ml). The combined organic phase was washed with 20% NaCl (446 ml) and then concentrated under vacuum at 40° C. to about 180 ml. The batch was dissolved with 600 ml of THF, concentrated under vacuum at 40° C. to about 180 ml and dissolved again with 600 ml of THF. Methanol (357 ml) was added followed by 35% aqueous hydrazine (512 ml). The batch was heated at reflux for over 1 hour, after which time the reaction was complete. The batch was cooled to 20-30° C., settled, and the lower layer split. The organic layer was concentrated under vacuum at 40° C. to about 180 ml, then mixed with 270 ml of EtOAc. The solution was washed 2 times with 20% NaCl (446 ml).

The batch was acidified with 270 ml of 2N HCl, extracting the product compound of Formula II-g into the lower acidic aqueous phase. The top phase was separated and the acidic aqueous phase was washed 10 times with 450 ml of EtOAc. The acidic phase containing the product was neutralized with 15% Na₃PO₄ and extracted with 300 ml of EtOAc and the organic layer was washed with 20% NaCl. The organic phase was then cooled to 0-10° C. and ethylisocyanate (EtNCO, 20.5 g) was added over 30 minutes. The solution was stirred another 30 minutes. To quench the reaction, 180 ml of 5% NH₄OH was added, followed by 270 ml of 20% NaCl. The aqueous layer was removed. The organic layer was washed again with 270 ml of 20% NaCl and then diluted with 2 L EtOAc. The batch was concentrated at atmospheric pressure to a volume of 270 ml and diluted again with 2 L EtOAc. This concentration/dilution sequence was repeated until a water content of 0.10% by KF titration is achieved. The batch was cooled to 0-10° C. and HCl gas (41 g) was charged subsurface over about 10 m during which time the product precipitated. At the end of the charge, the batch was agitated for about 30 minutes and then filtered. The wet cake was washed with EtOAc (267 ml) and then dried for 12 h in a vacuum oven at 60° C., yielding 125.7 g (73%) of an off-white solid: ¹H NMR (400 MHz, CD₃OD) δ 8.84 (s, 1H), 7.51 (m, 4H), 4.92 (m, 8H), 4.19 (m, 1H), 4.03 (m, 2H), 3.68 (m, 3H), 3.25 (m, 3H), 3.04 (bs, 1H), 2.66 (s, 3H), 2.26 (bs, 1H), 2.09 (bs, 2H), 1.92 (m, 1), 1.69 (m, 1H), 1.14 (m, 6H), 1.00 (t, 3H),; ¹³C NMR (100 MHz, CD₃OD) δ 171.7, 166.0, 161.3, 159.8, 150.0, 145.6, 140.9, 140.1, 137.6, 135.6, 130.5, 130.4, 130.3, 63.7, 61.4, 52.2, 52.1, 52.0, 51.1, 36.3, 23.5, 23.4, 21.8, 17.8, 16.2, 16.1, 10.9, 10.6; MSES+m/z (relative intensity) 571 (M+H).

### Preparation of Compound II-g (Optional)

For use in the above-described preparation of the compound of Formula IIh or the freebase of the compound of Formula IIh, the following procedure for the preparation of the compound of Formula II-g may optionally be selected. A mixture of Compound II-d-1 (10.0 g, 31.8 mmol), Compound II-e (10.3 g, 40.3 mmol) and sodium phosphate, tribasic (7.8 g, 47.6 mmol) in methyl acetate (75 ml) and water (75 ml) was heated to 35-45° C. until all solids were dissolved. The aqueous phase was removed in a separation funnel. The organic layer was washed with NaCl solution (20%, 50 ml) and concentrated under vacuum at 40° C. to a volume of about 20 ml. Tetrahydrofuran (80 ml) was charged and the batch was atmospherically concentrated to a volume of about 20 ml. Tetrahydrofuran (80 ml) was then charged. More distillation was allowed if necessary to meet the KF spec. The solution was then transferred to the mixture of sodium triacetoxyborohydride (10.8 g, 51.0 mmol) and sodium sulfate (22.6 g, 159 mmol). The batch was agitated at 20-30° C. for about 18 h. After reaction completion, water (100 ml) and 2-methyl tetrahydrofuran (30 ml) was added and the batch was heated to 35-45° C. to dissolve all solids. The batch was settled and split and washed with NaCl solution (20%, 50 ml). Methanol (40 ml) was added followed by aqueous hydrazine (35%, 57.4 ml). The batch was heated to reflux for about 1 hour. After reaction completion, the batch was cooled to 20-30° C., settled, and split. The organic layer was concentrated under vacuum at 40° C. to about 30 ml, diluted with 2-methyl tetrahydrofuran (60 ml), and washed with NaCl solution (20%, 50 ml) twice. The hydrochloride acid (2N, 30 ml) was charged and the batch was agitated for 3 minutes, settled and split. The aqueous phase was washed with 2-methyl tetrahydrofuran (50 ml) three times. The 2-methyl tetrahydrofuran (80 ml) and sodium phosphate solution (15%, 50 ml) were charged to the aqueous layer. The mixture was agitated for 3 minutes, settled and split. The organic layer was washed with sodium chloride solution (20%, 20 ml) and distilled under vacuum to about 40 ml. The batch was filtered, washed with 2-methyl tetrahydrofuran (30 ml) and dried under vacuum at 40° C. for about 18 hours to give 8.28 g (52.1%) white solid. ¹H NMR (400 MHz, in CDCl₃): 8.80 (S, 1H), 8.66 (S, 1H), 7.39 (m, 4H), 4.63 (br, 1H), 4.06 (br, 2H), 3.93 (m, 1H), 3.78 (br, 1H), 3.36 (dd, 1H), 3.17 (br, 1H), 3.07 (br, 1H), 2.91-2.79 (m, 4H), 2.51 (S, 3H), 2.47 (Br, 1H), 1.89 (br, 1H), 1.74 (br, 1H), 1.60-1.50 (br, 4H), 1.09 (d, 3H), 0.85 (t, 3H). ¹³C NMR (400 MHz, in CDCl₃): 169.5, 164.7, 159.7, 139.5, 136.0, 134.8, 129.1, 128.9, 57.8, 56.2, 51.5, 49.8, 49.2, 47.5, 41.9, 31.6, 30.5, 28.2, 26.8, 22.7, 19.2, 16.9, 11.0. ESI-MS: m/z 500.26 (M+H)⁺.

### Preparation of Compound II-G (Second Optional Example)

As mentioned above, preparation of the compound of Formula II-g may optionally be carried out using the following procedure, and then the product compound of Formula II-g employed in the preparation of the compound of Formula II-h in accordance with the above-discussed process. Accordingly, optionally, into a vessel containing 10.0 g the compound of Formula II-d-1, 9.4 g of the compound of Formula IIe and 80 mL 2-methyl tetrahydrofuran was added a solution of 3.3 g potassium carbonate and 7.5 g sodium chloride in 50 mL of water. After stirring at about 40° C. for 30 minutes, the organic layer was separated and dried via azeotropic distillation. The dry solution was transferred to a flask containing 22.6 g sodium sulfate (fine powder) and 11.5 g sodium triacetoxyborohydride. The resulting slurry was agitated at about 25° C. for about 24 hours and then 50° C. for about 6 hours before 0.1 g sodium boronhydride was added to reduce any remaining portion of the compound of Formula IIe. To dissolve all solids, about 80 mL of 40° C. water was added to the flask. Once all solids were dissolved, the organic layer was separated and then diluted with 40 mL methanol. Into the diluted organic layer was added 57.4 mL of 35 wt % aqueous hydrazine and the resulting mixture was heated to reflux and held at reflux for 4 hours yielding the compound of Formula II-g. The organic layer containing the compound of Formula II-g was separated and concentrated to about 30 mL, then diluted with 40 ml of 2-methyl tetrahydrofuran and washed with sodium chloride solution. After washing, the organic layer containing the compound of Formula II-g was extracted with 30 ml of 2N hydrochloric solution. After washing with 40 ml of 2-methyl tetrahydrofuran, the acidic aqueous layer containing the compound of Formula II-g was diluted with 50 ml of 2-methyl tetrahydrofuran and then basified with 15% potassium carbonate solution. The resulting organic solution was separated and dried via azeotropic distillation, providing an organic solution of about 40 ml volume which contained the compound of Formula II-g. This solution was heated to a temperature of 55° C. and slowly diluted with 10 ml of heptane, then seeded with 0.1 g of seed crystals of the compound of Formula II-g to initiate crystallization. The resulting slurry was agitated at 55° C. for 18 hours, and 20 mL of heptane was added portion wise. The slurry was cooled to about 20° C. and the resulting solids were filtered, washed and dried to give 11.1 g (70%) of light yellow crystals of the compound of Formula II-g.

Preparation of Compound II-h (Acid=H₃PO₄) from Compound II-g

Into a vessel containing 5 mL of water, 10.0 g of the compound of Formula II-g previously prepared and 60 ml of 2-methyl tetrahydrofuran was slowly added 1.6 g ethyl isocyanate while maintaining the reaction mixture temperature at 5° C. The reaction mixture was agitated for about 30 minutes and then quenched with 5% ammonium hydroxide solution. The organic layer was separated and washed twice with sodium chloride solution and dried via azeotropic distillation. The dried organic layer was diluted with 190 ml of acetone and heated to about 55° C. whereupon a solution of 4.4 g 85% phosphoric acid in 20 ml of acetone was slowly added to the hot organic layer. The resulting slurry was cooled to about 20° C., and the solids were filtered, washed and dried to give 13.7 g (90%) of off-white solid compound of the Formula II-h, where Acid=H₃PO₄. ¹HNMR (400 MHz, d₆-DMSO): 10.14 (br, 6H), 9.98 (s, 1H), 8.65 (s, 1H), 7.90 (s, 1H), 7.52 (d, 2H), 7.46 (d, 2H), 6.42 (t, 1H), 4.48 (br, 1H), 4.00 (br, 1H), 3.57 (br, 1H), 3.44 (d, 1H), 3.07 (m, 7H), 2.88 (br, 1H), 2.75 (br, 1H), 2.54 (s, 3H), 1.94 (br, 1H), 1.82 (br, 1H), 1.60 (m, 4H), 1.12 (d, 3H), 1.01 (t, 3H), 0.81 (br, 3H). ¹³CNMR (400 MHz, d₆-DMSO): 206.5, 167.7, 163.0, 157.9, 145.2, 138.6, 136.5, 134.9, 134.1, 128.8, 128.5, 66.7, 57.9, 56.5, 49.8, 34.0, 32.7, 30.7, 25.4, 21.7, 20.9, 16.3, 15.5, 10.1.

### Preparation of Compound A45

### Method A—Preparation of Compound A45 From Compound II-h (Acid=HCl)

A mixture of Compound II-h (Acid=HCl) previously prepared (10.0 g, 16.5 mmol), potassium carbonate (17.4 g, 125.9 mmol), 4-chlorobenzene-sulfonylchloride (6.6 g, 31.3 mmol) and 4-N,N-dimethylamino pyridine (0.18 g, 1.5 mmol) in tetrahydrofuran (30 ml) and acetonitrile (50 ml) was agitated between 20 and 30° C. for at about 3 hours. Tetrahydrofuran (35 ml), ammonium hydroxide solution (28-30%, 5 ml) followed by water (45 ml) were added. The batch was heated up to dissolve the remaining solid, agitated at 45 to 55° C. for 3 hours, settled for 30 minutes and split. The organic layer was atmospherically concentrated to about 40 ml and acetonitrile (60 ml) was charged. The batch was concentrated atmospherically to 40 ml, cooled to 20° C. and agitated for about 30 minutes. The batch was then filtered and washed with acetonitrile (40 ml) and water (120 ml). The wet cake was dried under vacuum at 65-75° C. for about 12 hours, to give 7.5 g (83%) light yellow solid. ¹H NMR (400 MHz, in CDCl₃): 8.78 (S, 1H), 7.39 (m, 4H), 5.04 (t, 1H), 4.66 (br, 1H), 3.86 (br, 2H), 3.52 (m, 2H), 3.32 (dd, 1H), 3.08 (b, 2H), 2.92 (t, 3H), 2.75 (b, 1H), 2.59 (s, 3H), 2.47 (br, 1H), 1.90 (b, 1H), 1.73 (b, 1H), 1.56 (m, 4H), 1.32 (t, 3H), 1.08 (d, 3H), 0.84 (t, 3H). ¹³C NMR (400 MHz, CDCl₃): 170.9, 165.7, 159.5, 158.6, 148.9, 139.6, 137.3, 136.2, 133.5, 130.5, 130.3, 59.2, 57.5, 53.1, 51.6, 49.0, 43.3, 40.2, 33.0, 32.0, 29.1, 27.9, 23.8, 21.1, 18.3, 16.7, 12.3. ESI-MS: m/z 553.1 (M+H)⁺.

### Method B—Preparation of Compound A45 From Compound II-h (Acid=HCl)

To a suspension of Compound II-h (Acid=HCl) previously prepared (5.0 g, 8.2 mmol) and 4-N,N-dimethylamino pyridine (0.10 g, 0.82 mmol) in tetrahydrofuran (30 ml) was charged triethylamine (4.59 ml, 32.9 mmol). The mixture was agitated between 20 and 30° C. for about 30 minutes. The diethylphosphoric chloride (2.36 ml, 16.5 mmol) was then charged. The batch was agitated between 20 and 30° C. for about 30 minutes. The ammonium hydroxide solution (28-30%, 2.5 ml) and sodium chloride solution (15%, 10 ml) were added and agitated at about 40° C. for 30 minutes. The aqueous phase was removed in a separation funnel. A solution of potassium carbonate (2.88 g, 20.8 mmol) in water (11 ml) was charged to the organic layer. The mixture was refluxed for about 24 hours and then cooled to 40° C., settled and split. Acetonitrile (45 ml) was charged and the batch was distilled to about 20 ml. Water (2.5 ml) was charged and the batch was refluxed for about 1 hour and cooled to 20 to 30° C. The batch was filtered, washed with mixture of acetonitrile and water (9:1, 20 ml) and dried under vacuum at about 30° C. for 5 hours, to give 3.82 g (83.9%) light yellow solid.

### Method C—Preparation of Compound A45 From Compound II-h (Acid=H₃PO₄)

To a mixture of 10.0 g of the compound of Formula II-h (acid=H₃PO₄) previously prepared, 70 ml of 2-methyl tetrahydrofuran and 25 ml of water was added 25 ml of 20% potassium carbonate solution. The mixture was agitated for 15 minutes and the organic layer was separated and washed with 15 ml of 10% sodium chloride solution. Water was removed from the batch using a Dean-Stark trap via azeotropic distillation. Following drying, 50 mL of acetonitrile, 7.0 g of potassium carbonate (extra fine powder), 5.2 g of 4-chlorobenzenesulfonyl chloride and 0.16 g of dimethylaminopyridine was added to the reaction mixture. The mixture was agitated while maintaining the temperature of the reaction mixture between 20° C. and 30° C. for 3 hours. The mixture was then quenched with 25 mL of 2-Methyl tetrahydrofuran, 50 mL of water and 5 mL of ammonium hydroxide solution (28-30%). The resulting mixture was stirred at 50° C. for one hour. The aqueous layer was removed and the organic layer was concentrated atmospherically to a volume of 40 ml. Acetonitrile (60 ml) was added and the solution was concentrated to 40 ml again to result in a suspension. The mixture was cooled to room temperature and the solids were collected by filtration, to give 5.8 g (80%) of off-white crystals of the compound of Formula A45.

### Preparation of Compound II-1

### Method A:

To a suspension of 10 g (18.1 mmol) of Compound A45 in a mixture of 140 ml of THF, 8 ml of 1-methyl-2-pyrrolidinone and 1.0 ml of water was added 85 mg of methanesulfonic acid. The mixture was heated up to 60° C. After being stirred for 5 minutes at 60° C., the resulting solution was cooled to room temperature. The batch was then filtered to remove any insoluble solids. The filtrate was heated to 60° C. and 10 mg of Compound II-1 seeds were charged. A solution of 1.64 g of methanesulfonic acid in 10 ml of THF was charge thorough an additional funnel over 3 hours with vigorous agitation at 60° C. The resulting suspension was cooled to room temperature over 30 minutes. The solids were collected by filtration and washed with 20 ml of THF followed by 20 ml of heptane. The wet cake was dried in a vacuum over at 85° C. for 12 h, to give 10.4 g (89%) of white solids. ¹H NMR (400 MHz, in CD₃OD): 8.80 (S, 1H), 7.52 (m, 4H), 4.85 (d, 1H), 4.00 (br, 2H), 3.91 (d, 1H), 3.6-3.8 (br, 3H), 3.92 (q, 2H), 3.85 (br, 1H), 3.72 (br, 1H), 2.95 (m, 2H), 2.71 (s, 3H), 2.63 (d, 3H), 2.22 9d, 1H), 2.0-2.15 (br, 3H), 1.95 (br, 1H), 1.85 (br, 1H), 1.30 (t, 3H), 1.11 (d, 3H), 0.97 (q, 3H). ¹³C NMR (400 Mhe, CD₃OD): 171.3, 165.6, 157.7, 157.6, 151.8, 139.1, 137.3, 135.6, 135.0, 130.0, 129.9, 62.8, 60.0, 52.9, 51.9, 51.3, 47.3, 41.7, 39.6, 38.9, 28.5, 25.6, 22.3, 20.8, 17.2, 14.8, 9.9. ESI-MS: m/z 553.3 (M+H)⁺. Anal. Calcd. for C₂₉H₄₁CIN₈O₅S (649.20): C, 53.64; H, 6.37; N, 17.25; S, 4.93; CI, 5.47. Found: C, 53.51; H, 6.18; N, 17.10; S, 4.94; CI, 5.65.

### Method B:

To a suspension of 10.0 g (1×) of Compound A45 in 160 ml (16×) of acetone was added 1.7 g (0.17×) of methanesulfonic acid in 5.0 ml (0.5×) of water at room temperature. The mixture was stirred for 30 min at ambient temperature and the resulting solution was filtered to remove insoluble particles. The solution was then seeded with small amount of Compound II-1 crystals and the volume of the batch was reduced to 100 ml (10×) by distillation under one atmosphere. During the distillation, crystalline solids precipitate from the solution. To the suspension at 60° C., 170 ml (17×) of THF was charged and the volume of the resulting mixture was reduced again to 100 ml (10×) by distillation. The mixture was sampled for water content by KF analysis. If the water content is above 1.2%, 20 ml (2×) THF was charged and the batch was concentrated to 100 ml (10×) again. This procedure was repeated until the water content dropped below 1.2%. The batch was filtered after being cooled to room temperature. The cake was washed with THF and dried in a vacuum oven for 12 h at 70° C., to give 10.3 (88%) white solids.

### Preparation of Compound III-a

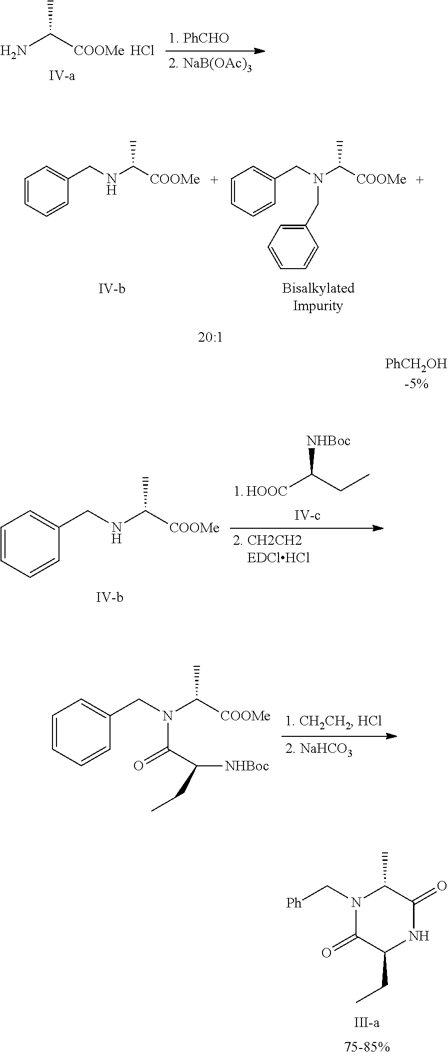

### Conversion of D-Analine Methylester Hydrochloride to Compound IV-b:

To a solution of 25.0 g (0.18 M) of D-analine methylester hydrochloride and 18 ml (0.72×) of benzaldehyde in 500 ml (20×) of methylene chloride was added 45.5 g (1.82×) of NaBH(OAc)₃ slowly in about 15 portions over 90 minutes at a temperature between 20 and 25° C. 100 ml (4×) of 1 M HCl solution and 75 ml (3×) of water were added after the mixture was stirred at room temperature for an additional hour. The mixture was stirred for 5 minutes and the aqueous layer was separated. The organic phase was discarded. The aqueous layer contained essentially pure product Compound IV-b as HCl salt while the bisalkylated impurity did not form HCl salt. This impurity remained in organic phase along with by-product benzyl alcohol. Isopropyl acetate (125 ml, 5×) was added to the aqueous layer and the mixture was neutralized with 25% sodium hydroxide solution until the pH reached about 10. The organic layer was separated and the aqueous layer extracted with 125 ml (5×) of isopropyl acetate. The combined organic layers were evaporated to dryness under vacuum to give 28.6 g oil. The analysis showed that the purity of this material is about 95% and molar yield is 79%. The amount of enantiomeric isomer by chiral HPLC is 1.9%.

### Conversion of Compound IV-b to Compound III-a:

To a solution 20.0 g (0.103 mol, 1×) of Compound IV-b and 23.2 g (1.16×, 1.13 eq.) of N-Boc-2-(S)-aminobutyric acid in 160 ml (8×) of methylene chloride was added 11 g (0.55×, 0.55 eq.) of EDCl.HCl at room temperature. The mixture was stirred one hour at ambient temperature and another 11 g (0.55×, 0.55 eq.) of EDCl.HCl was charged in two equal portions over 30 minutes. After the mixture was stirred for an additional hour, the batch was analyzed by HPLC and additional EDCl.HCl could be charged in small quantities until a conversion over 96% was reached. The reaction mixture was then mixed with 160 ml (8×) of 0.5 N HCl solution and 10 g (0.5×) of Celite. The organic layer was separated after the mixture was filtered. The organic layer was then neutralized with sodium bicarbonate and aqueous layer was removed. Hydrogen chloride gas was then bubbled through the organic layer at room temperature for about 3 hours until the Boc-deprotection reaction was complete. The acidic solution was then neutralized with saturated sodium bicarbonate to a pH of about 8 to initiate the cyclization reaction. The organic layer containing Compound III-a was then separated and the aqueous layer extracted with 30 ml (1.5×) of methylene chloride. The solvent was removed under vacuum and the residue crystallized from a mixture of 60 ml of tert-butyl methylether and 40 ml of heptane, resulting in 25.6 g (79%) white solids, de 97%. H NMR (400 MHz, in CDCl₃): 7.3 (m, 5H), 6.8 (s, 1H), 5.28 (d, 1H, J=14.9 Hz), 4.08 (m, 1H), 4.00 (d, 1H, J=14.9 Hz), 3.85 (q, 1H), 2.03 (m, 2H), 1.45 (d, 3H), 0.99 (t, 3H).

### Preparation of Compound II-E

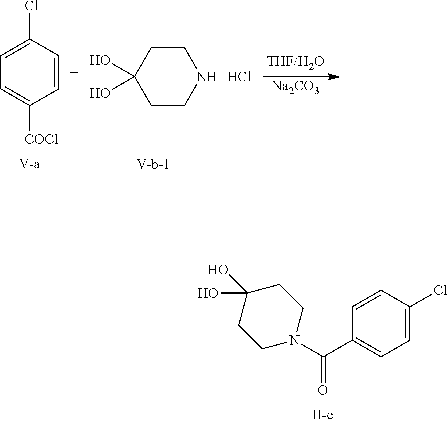

To a mixture of 20.0 g (0.13 mol) of Compound V-b-1 in 70 ml of water and 90 ml of tetrahydrofuran was added 27.3 g (0.156 mol, 1.2 eq.) of Compound V-a below 5° C. The mixture was cooled to −10° C. and a solution of 2.6 equivalents of 2M aqueous Na₂CO₃ was over about 90 minutes while maintaining batch temperature below 10° C. During the addition, the product precipitated. After agitation for about 1 hour at 10° C., the reaction mixture was warmed up to about 20° C. followed by addition of 200 ml of water over 30 minutes. The resulting suspension was cooled back to 10° C. and filtered. The wet cake was washed repeatedly with water and dried in a vacuum oven at 25° C. for about 24 hours, to give 30.0 g (90%) white powders. Karl Fisher analysis of the powders showed 7.0% of water as monohydrate. The HPLC analysis showed the purity of the product is greater than 99%. ¹H NMR (400 MHz in DMSO): 7.48 (dd, 4H), 3.61 (br, 2H), 3.26 (br, 2H), 1.75 (br, 2H), 1.52 (br, 2H).

The above description of the invention is intended to be illustrative and not limiting. Various changes or modifications in the embodiments described herein may occur to those skilled in the art. These changes can be made without departing from the scope or spirit of the invention

1. A process for preparing a compound of the Formula A45: 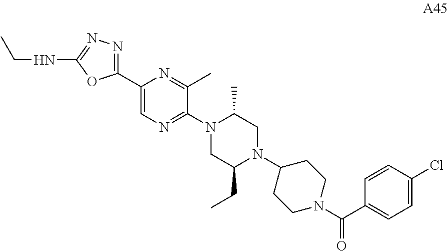
or a physiologically acceptable salt, solvate or prodrug thereof, said process comprising:
a) providing a compound of the Formula II-a: 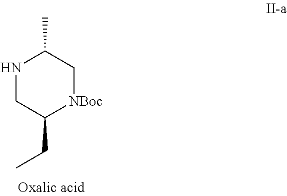
wherein Boc represents a tert-butoxycarbonyl protective group;
b) reacting the free base form of the compound of the Formula II-a with the compound of the Formula II-b: 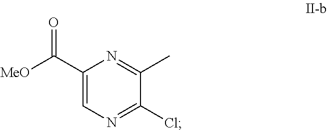
to form the compound of the Formula II-c: 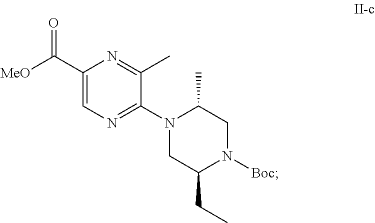
c) deprotecting the compound of the Formula II-c by treating with an acid to form a salt form of the compound of the Formula II-d: 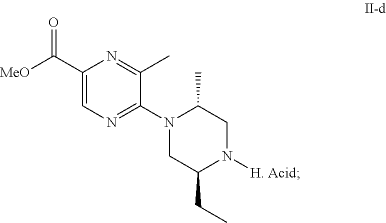
d) coupling the compound of the Formula II-d with the compound of the Formula II-e: 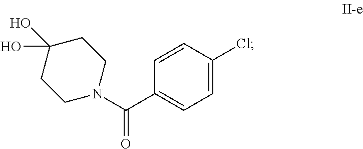
to form the compound of the Formula A44: 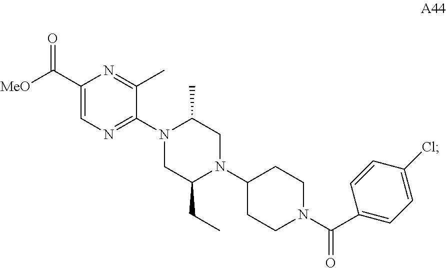
e) reacting the compound of the Formula A44 with hydrazine (NH₂NH₂) to form the compound of the Formula II-g: 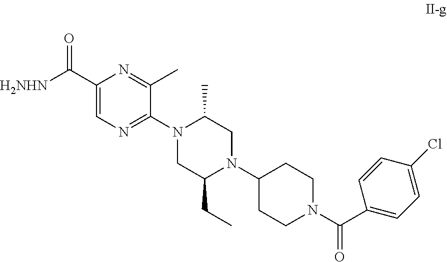
and optionally isolating the compound of Formula II-g;
f) reacting the compound of the Formula II-g with Ethylisocyanate (EtNCO) then hydrochloric acid to form the compound of the Formula II-h: 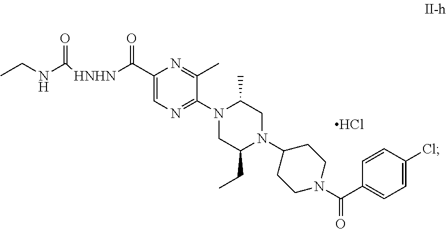
g) cyclizing the hydrazide functionality of the compound of the Formula II-h to form the compound of the Formula A45: 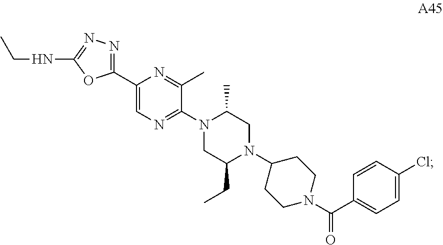
and
h) optionally converting the compound of the Formula A45 to a physiologically acceptable salt, solvate or prodrug thereof.

2. The process according to claim 1, wherein the compound of the Formula II-a: 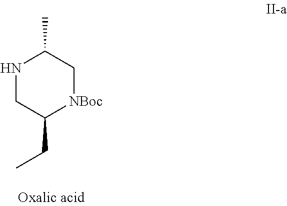
is prepared by a process that comprises reacting the compound of the Formula III-a: 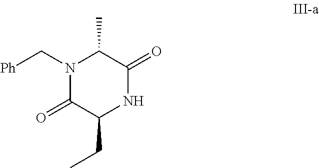
with lithium aluminum hydride and then di-tert-butyl dicarbonate to form the compound of the Formula III-b: 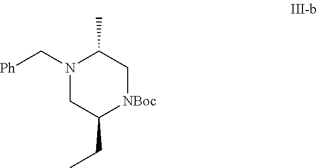
and then hydrogenating the compound of the Formula III-b and treating the resulting product with oxalic acid to yield the compound of the Formula II-a.

3. The process according to claim 2, wherein the compound of Formula III-a: 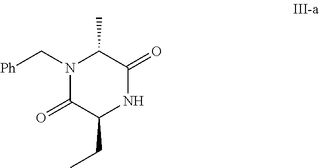
is prepared by a process that comprises coupling the compound of the Formula IV-a: 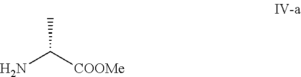
or a salt thereof with benzaldehyde (PhCHO) to form the compound of the Formula IV-b: 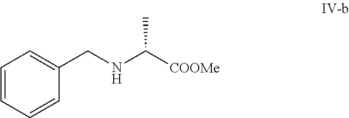
and then reacting the compound of the Formula IV-b with a compound of the Formula IV-c: 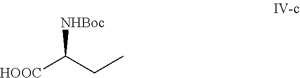
and deprotecting then cyclizing the deprotected product to form the compound of the Formula III-a.

4. The process according to claim 3, which comprises coupling the hydrochloride salt of the compound of Formula IV-a with benzaldehyde in the presence of NaB(OAc)₃.

5. The process of claim 4, wherein the reacting of the compound of Formula IV-b with the compound of Formula IV-c takes place in 1-(3-dimethylaminopropyl)-3-ethyl-carbodimide hydrochloride said deprotecting is carried out in hydrochloric acid, said ring-closing is carried out in sodium bicarbonate solution and where Step C comprises deprotecting the compound of the Formula IIc by treatment with hydrochloric acid in solution in admixture with 2-isopropanol.

6.-8. (canceled)

9. The process of claim 3 wherein the compound of the Formula II-e: 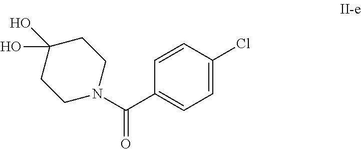
is prepared by a process that comprises reacting the compound of the Formula V-a: 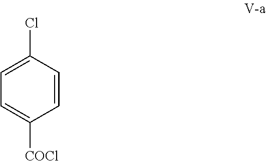
with a compound of the Formula V-b:

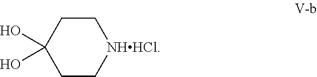

10. The process according to claim 9, wherein said reacting is conducted in the presence of potassium carbonate, and the compound of the Formula II-e precipitates from the reacting mixture as a monohydrate.

11. (canceled)

12. The process of claim 10, wherein step f) comprises reacting the compound of the Formula A44 with an aqueous solution of the hydrazine.

13. (canceled)

14. The process of claim 9 wherein Step (h) is carried out and comprises converting the compound of the Formula A45 to a physiologically acceptable salt thereof.

15. The process of claim 14 wherein Step (h) comprises reacting the compound of Formula A45 with methanesulfonic acid to form the methanesulfonate salt of the compound of Formula A45.

16. A compound selected from the group consisting of the compound of the Formula II-g: 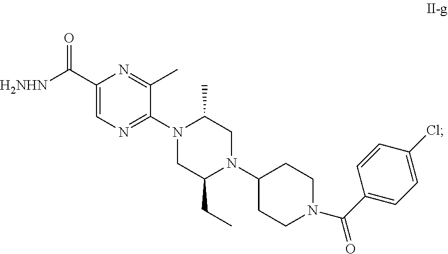
and
the compound of the Formula II-h:

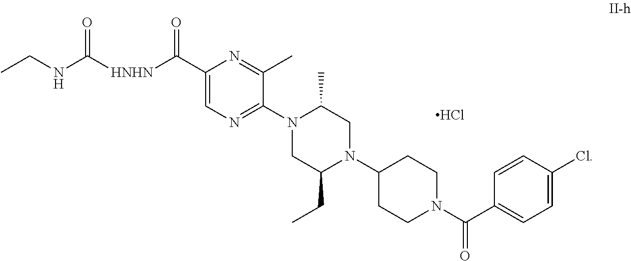

17. The compound of the Formula II-1:

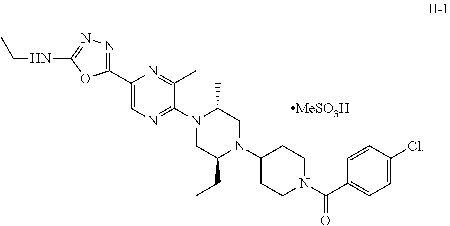

18. A pharmaceutical formulation comprising the salt of claim 17.

19. The compounds of Formula II-c and II-d:

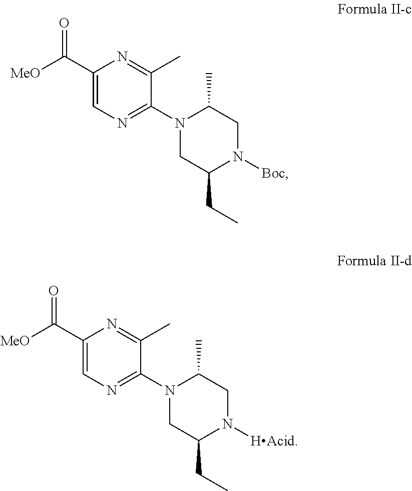

20. The compounds, of Formula II-a and II-e: [chemical structure]
